(19) United States
(12) Patent Application Publication
(10) Pub. No.: US 2008/0268443 A1
(43) Pub. Date: Oct. 30, 2008

(54) BROAD-BASED DISEASE ASSOCIATION FROM A GENE TRANSCRIPT TEST

(75) Inventor: Dean Iverson Sproles, Seattle, WA (US)

Correspondence Address: Graybeal Jackson Haley LLP / Jablonski Law Group, 155 - 108th Ave NE Suite 350, Bellevue, WA, 98004, US

(73) Assignee: IGD INTEL, LLC, Seattle, WA (US)

(21) Appl. No.: 11/756,864

(22) Filed: Jun. 1, 2007

Related U.S. Application Data
(60) Provisional application No. 60/913,755, filed on Apr. 24, 2007.

(51) Int. Cl. C12M 1/34; C12Q 1/68

(52) U.S. Cl. 435/6; 435/287.2; 435/288.7

(57) ABSTRACT

Broad-based disease association gene transcript test and data structure. Disease considerations for this unique test include a custom set of genetic sequences associated in peer-reviewed literature with various known diseases such as Addison's disease, anemia, asthma, atherosclerosis, autism, breast cancer, estrogen metabolism, Grave's disease, hormone replacement therapy, major histocompatibility complex (MHC) genes, longevity, lupus, multiple sclerosis, obesity, osteoarthritis, prostate cancer, and type 2 diabetes. The base dataset may be developed through clinical samples obtained by third-parties. Online access of real-time phenotype/genotype associative testing for physicians and patients may be promoted through an analysis of a customized microarray testing service.

### CROSS-REFERENCE TO PROVISIONAL PATENT APPLICATION

This patent application claims priority from a related provisional patent application entitled ‘BROAD-BASED DISEASE ASSOCIATION GENE TRANSCRIPT TEST’ filed on Apr. 24, 2007 which is incorporated herein in its entirety.

### BACKGROUND

Genetic diseases afflict many people and remain the subject of much study and misunderstanding. Some genetic disorders may be caused by the abnormal chromosome number, as in Down syndrome (extra chromosome 21) and Klinefelter's syndrome (a male with 2X chromosomes). Triplet expansion repeat mutations can cause fragile X syndrome or Huntington's disease, by modification of gene expression or gain of function, respectively. Other genetic disorders occur when specific gene sequences are not maintained as expected, such as with Multiple Sclerosis and Type II diabetes. Currently, around 4,000 genetic disorders are known, with more being discovered as more is understood about the human genome. Most disorders are quite rare and affect one person in every several thousands or millions while other are more common such as cystic fibrosis wherein about 5% of the population of the United States carry at least one copy of the defective gene.

A person's genetic makeup is reflected through Deoxyribonucleic Acids (DNA). DNA is a molecule that comprises sequences of nucleic acids (i.e., nucleotides) that form the code which contains the genetic instructions for the development and functioning of living organisms. A DNA sequence or genetic sequence is a succession of any of four specific nucleic acids representing the primary structure of a real or hypothetical DNA molecule or strand, with the capacity to carry information. As is well understood in the art, the possible nucleic acids (letters) are A, C, G, and T, representing the four nucleotide subunits of a DNA strand—adenine, cytosine, guanine, and thymine bases covalently linked to phospho-backbone. Typically the sequences are printed abutting one another without gaps, as in the sequence AAAGTCTGAC. A succession of any number of nucleotides greater than four may be called a sequence.

Ribonucleic acid (RNA) is a nucleic acid polymer consisting of nucleotide monomers, that acts as a messenger between DNA and ribosomes, and that is also responsible for making proteins by coding for amino acids. RNA polynucleotides contain ribose sugars unlike DNA, which contains deoxyribose. RNA is transcribed (synthesized) from DNA by enzymes called RNA polymerases and further processed by other enzymes. RNA serves as the template for translation of genes into proteins, transferring amino acids to the ribosome to form proteins, and also translating the transcript into proteins.

A gene is a segment of nucleic acid that contains the information necessary to produce a functional product, usually a protein. Genes contain regulatory regions dictating under what conditions the product is produced, transcribed regions dictating the structure of the product, and/or other functional sequence regions. Genes interact with each other to influence physical development and behavior. Genes consist of a long strand of DNA (RNA in some viruses) that contains a promoter, which controls the activity of a gene, and a coding sequence, which determines what the gene produces. When a gene is active, the coding sequence is copied in a process called transcription, producing an RNA copy of the gene's information. This RNA can then direct the synthesis of proteins via the genetic code. However, RNAs can also be used directly, for example as part of the ribosome. These molecules resulting from gene expression, whether RNA or protein, are known as gene products.

The total complement of genes in an organism or cell is known as its genome. The genome size of an organism is loosely dependent on its complexity. The number of genes in the human genome is estimated to be just under 3 billion base pairs and about 20,000-25,000 genes.

As previously mentioned, certain genetic disorders may result from DNA sequences being incorrectly coded. A Single Nucleotide Polymorphism or SNP (often time called a “snip”) is a DNA sequence variation occurring when a single nucleotide—A, T, C, or G—in the genome (or other shared sequence) differs between members of a species (or between paired chromosomes in an individual). For example, two sequenced DNA fragments from different individuals, AAGCCTA to AAGCTTA, contain a difference in a single nucleotide. In this case, this situation may be referred to as having two alleles: C and T.

Within a population, Single Nucleotide Polymorphisms can be assigned a minor allele frequency—the ratio of chromosomes in the population carrying the less common variant to those with the more common variant. Usually one will want to refer to Single Nucleotide Polymorphisms with a minor allele frequency of ≧1% (or 0.5% etc.), rather than to “all Single Nucleotide Polymorphisms” (a set so large as to be unwieldy). It is important to note that there are variations between human populations, so a Single Nucleotide Polymorphism that is common enough for inclusion in one geographical or ethnic group may be much rarer in another.

Single Nucleotide Polymorphisms may fall within coding sequences of genes, noncoding regions of genes, or in the intergenic regions between genes. Single Nucleotide Polymorphisms within a coding sequence will not necessarily change the amino acid sequence of the protein that is produced, due to degeneracy of the genetic code. A Single Nucleotide Polymorphism in which both forms lead to the same polypeptide sequence is termed synonymous (sometimes called a silent mutation)—if a different polypeptide sequence is produced they are non-synonymous. Single Nucleotide Polymorphisms that are not in protein coding regions may still have consequences for gene splicing, transcription factor binding, or the sequence of non-coding RNA.

Variations in the DNA sequences of humans can affect how humans develop diseases, and/or respond to pathogens, chemicals, drugs, etc. However, one aspect of learning about DNA sequences that is of great importance in biomedical research is comparing regions of the genome between people (e.g., comparing DNA sequences from similar people, one with a disease and one without the disease). Technologies from Affymetrix™ and Illumina™ (for example) allow for genotyping hundreds of thousands of Single Nucleotide Polymorphisms for typically under $1,000.00 in a couple of days.

Microarray analysis techniques are typically used in interpreting the data generated from experiments on DNA, RNA, and protein microarrays, which allow researchers to investigate the expression state of a large number of genes—in many cases, an organism's entire genome—in a single experiment. Such experiments generate a very large volume of genetic data that can be difficult to analyze, especially in the absence of good gene annotation. Most microarray manufacturers, such as Affymetrix™, provide commercial data analysis software with microarray equipment.

Specialized software tools for statistical analysis to determine the extent of over- or under-expression of a gene in a microarray experiment relative to a reference state may aid in identifying genes or gene sets associated with particular phenotypes. Such statistical packages typically offer the user information on the genes or gene sets of interest, including links to entries in databases such as NCBI's GenBank and curated databases such as Biocarta and Gene Ontology.

As a result of a statistical analysis, specific aspects of an organism may be genotyped. Genotyping refers to the process of determining the genotype of an individual with a biological assay. Current methods of doing this include PCR, DNA sequencing, and hybridization to DNA microarrays or beads. The technology is intrinsic for tests on father-/motherhood and in clinical research for the investigation of disease-associated genes.

The phenotype of an individual organism is either its total physical appearance and constitution or a specific manifestation of a trait, such as size, eye color, or behavior that varies between individuals. Phenotype is determined to a large extent by genotype, or by the identity of the alleles that an individual carries at one or more positions on the chromosomes. Many phenotypes are determined by multiple genes and influenced by environmental factors. Thus, the identity of one or a few known alleles does not always enable prediction of the phenotype.

In a drawback of the current state of the art, the genotyping process is typically accomplished for a single patient or research sample in a single sampling for a single iteration and with a specific disease in mind for the genotyping. As such, the results are relatively isolated with respect to any possible comparison and analysis of other similarly situated patients. Furthermore, such isolation leads to inefficiencies in diagnostics and treatment of the underlying results of the test. Without a system for allowing the sharing of underlying data, all potential benefits of aggregating the data are lost. Thus, as genetic material samples are collected, they are done so from an individualistic approach without regard for benefits to be realized from aggregating the data from many genetic samples from many sample sources (i.e., people). What is needed is a broad-based disease association gene transcript test along with systems and methods associated therewith capable of allowing the assimilation of a wide range of data from a wide range of sources.

### BRIEF DESCRIPTION OF THE DRAWINGS

The foregoing aspects and many of the attendant advantages of the claims will become more readily appreciated as the same become better understood by reference to the following detailed description, when taken in conjunction with the accompanying drawings, wherein:

FIG. 1 shows a diagram of a method for preparing a microarray to be used in a broad-based disease association gene transcript test according to an embodiment of an invention disclosed herein;

FIG. 2 shows a diagrammatic representation of a method for collecting genetic material samples from several sources and detecting and isolating strands of genetic material for grouping according to an embodiment of an invention disclosed herein;

FIG. 3 is a diagrammatic representation of a system and method for establishing a data structure to be used in a broad-based disease association gene transcript test according to an embodiment of an invention disclosed herein;

FIG. 4 shows a typical arrangement of data that may be associated in a database of information derived from a broad-based disease association gene transcript test according to an embodiment of an invention disclosed herein; and

FIG. 5 shows a diagrammatic representation of a method and system for establishing a broad-based disease association gene transcript test according to an embodiment of an invention disclosed herein.

### DETAILED DESCRIPTION

The following discussion is presented to enable a person skilled in the art to make and use the subject matter disclosed herein. The general principles described herein may be applied to embodiments and applications other than those detailed above without departing from the spirit and scope of the present detailed description. The present disclosure is not intended to be limited to the embodiments shown, but is to be accorded the widest scope consistent with the principles and features disclosed or suggested herein.

The subject matter disclosed herein is related to transcriptional detection of single nucleotide polymorphisms (SNP) and insertion/deletion (I/D) genetic polymorphisms through a proportional analysis of RNA sequences detected through fluorescence hybridization on a custom manufactured microarray gene expression platform. SNPs may be identified through a specific design method (SNPs are typically assessed through DNA analysis). Disease considerations for this unique test include a custom set of genetic sequences associated in peer-reviewed literature with various known diseases such as Addison's disease, anemia, asthma, atherosclerosis, autism, breast cancer, estrogen metabolism, Grave's disease, hormone replacement therapy, major histocompatibility complex (MHC) genes, infectious disease screening panel, longevity, lupus, multiple sclerosis, obesity, osteoarthritis, prostate cancer, and type 2 diabetes. The base dataset may be developed through clinical samples obtained by third-parties clinical groups, and in partial association with the Swank MS Foundation. Further, coordination and volunteer efforts from followers of the Swank Program, as defined in the Multiple Sclerosis Diet Book (authored by Roy L. Swank) may be assimilated and utilized. Online access of real-time phenotype/genotype associative testing for physicians and patients may be promoted through a testing service.

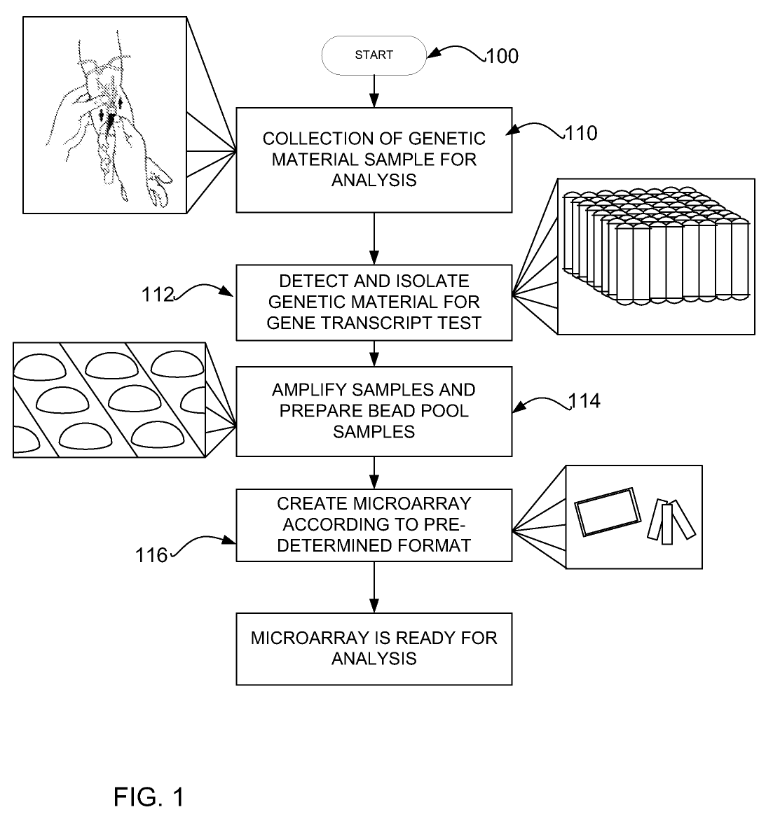

Various embodiments and methods of new processes include the assembly and association of genetic material samples with associated diseases, the preparation of microarrays with representative genetic material samples in a pattern best suited for analysis as well as manipulation, and delivery of assimilated and compiled data across a computer network. Various aspects of these embodiments are discussed in FIGS. 1-5 below.

FIG. 1 shows a diagram of an overall method 100 for preparing a data structure (e.g., a microarray) that may be used in a broad-based disease association gene transcript test according to an embodiment of an invention disclosed herein. The method may typically include drawing a blood sample (or obtaining another source of genetic material) from a patient scheduled for genotyping in step 110. Of course, in order to assimilate a broad-based set of data across several diseases, blood samples are typically drawn from several sources. It should be noted that any tissue suitable for gaining access to genetic material (e.g., DNA and/or RNA) may be used, such as liver tissue. Blood cells are easily collected and easily transported making this source for DNA/RNA efficient and effective. The blood sample may typically be collected using a suitable blood collection device such as blood collection tubes that are available from Paxgene™.

The sample is typically properly tagged and labeled by an anonymous yet traceable patient identification. That is, all measures are taken to comply with the Health Insurance Portability and Accountability Act (HIPAA) such that the blood sample is identifiable but also protected from accidental disclosure of privileged information. At the time of collection, additional demographic information may be stored (e.g., written on a tag, stored in a computer database) with the blood sample. Such demographic information may include a number of different descriptive phenotypic characteristics, such as age, sex, country of origin, race, specific health issues, occupation, birthplace, current living location, etc.

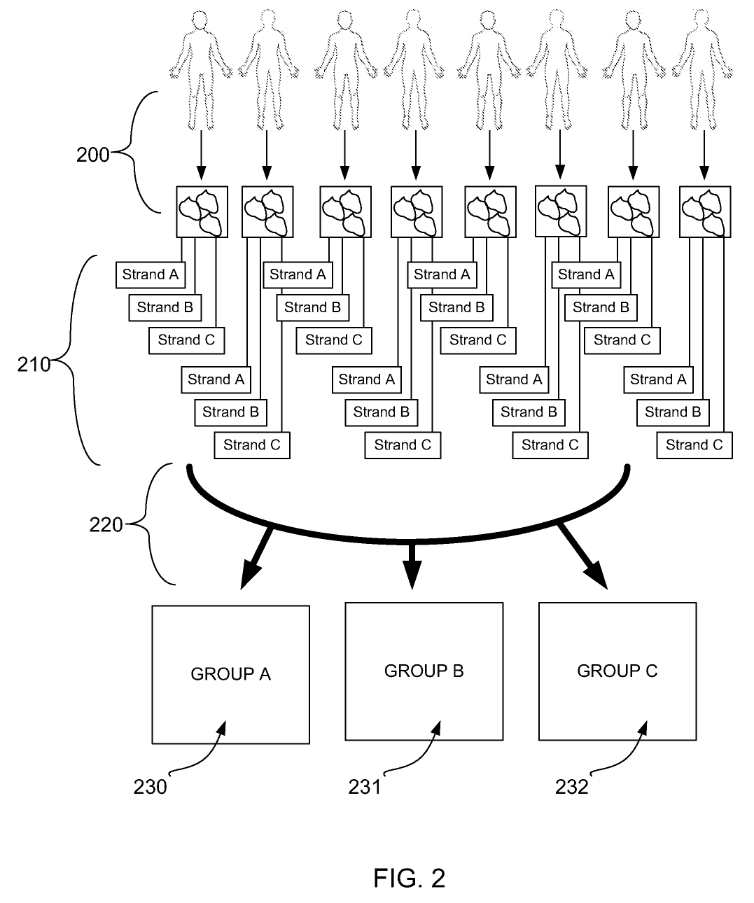

Specific genetic material, such as RNA from the blood sample, may then be detected and isolated in step 112 using an RNA isolation kit such as those that are available from Qiagen™. As mentioned above, RNA isolation may be accomplished at the same physical location as collection or may be accomplished at a remote laboratory after collection. The genetic material isolation process is described in more detail below with respect to FIG. 2.

At step 114, specific sequences in an RNA sample may be amplified using a fluorescence process that may be specific to pre-determined strands of RNA such as available from Illumina™ in a product entitled DASL™. In an alternative embodiment, specific sequences in DNA may also be amplified using a similar fluorescence process that may be specific to pre-determined strands of DNA such as available from lllumina™ in a product entitled Golden Gate™.

The isolation of genetic materials is typically followed by amplification of fluorescently labeled copies that may then be hybridized to specific probes attached to a common substrate, i.e., a microarray. However, the collected and isolated samples may be arranged and analyzed in any data structure suitable for analysis. As such, data may be collected and assimilated directly into a computer-based data structure, such as a database.

At step 116, the isolated and amplified samples of genetic material may be grouped according to identified sets of strands of genetic material. The groups may be arranged in a specific pattern in bead pools on a microarray according to a predetermined format. Such predetermined formats may include a standard format suitable for individual analysis of all identified genes in isolated RNA/DNA strands. Other predetermined formats may include a side-by-side comparison to one or more control groups of similar genes from control group samples. Other formats may include specific sets of genes suitable for broad-based disease association, multiple sclerosis association, broad-based diagnostics collection, broad-based predictive treatment data sets, or any other association of genes with samples. Once the microarray has been created in a specific pattern, the emergence of patterns and the like may be ready for analysis at step 118. The preparation of each microarray is described in more detail in U.S. patent application Ser. No. ______ entitled, “Method and System for Preparing a Microarray for a Disease Association Gene Transcript Test,” assigned to IGD-Intel of Seattle, Wash., which is incorporated by reference. The formats for arranging samples in a microarray typically follow specifics associated with the groupings of blood samples as discussed below with respect to FIG. 2.

FIG. 2 shows a diagrammatic representation of a method for collecting blood samples from several sources and identifying strands of genetic material for grouping according to an embodiment of an invention disclosed herein. In an overview of one method disclosed herein, one may begin the method by collecting a plurality of similar blood samples from a plurality of similar sources, the blood samples suitable for genetic code isolation and analysis. Then, identifiable strands of genetic material in each blood sample may be detected and isolated such that the strands of genetic material identifiable by a gene sequence or nucleotide sequence.

Next, for each blood sample, as an identifiable strand emerges, the samples may be separated into sets of samples with similar identifiable strands and then each set of isolated strand samples of genetic materials may be then grouped into groups of genetic material from each of the plurality of blood samples, such that each group comprises similar identifiable strands of genetic material from each blood sample. Once grouped, each group of genetic material maybe associated with a disease relevant to the identifiable strands comprising each group or any other relevant data that may be useful for diagnostics. Aspects of these broad-based steps are discussed below.

In FIG. 2, several different sources of genetic material may typically be used to obtain several different samples of genetic material. This step is represented in the aggregate at step 200 in FIG. 2 and may be associated with the individual step 110 of FIG. 1. As a result, several different and identifiable samples of genetic material may then be processed to detect and isolate specific genetic material for assimilation into an aggregate context. One such process includes RNA isolation.

Specific gene sequences (i.e., nucleotide sequences) may be identified when detecting and isolating strands of genetic material from each sample at step 210. On an aggregate level, each sample may typically have a first strand, such as STRAND A, such that all gene sequences that may be identified as STRAND A may be isolated and the sample separated from all other strands. Likewise, STRAND B for each sample may be also isolated and its respective sample separated. The case is also the same for STRAND C and every other identifiable strand of genetic material in each sample. Although, only 3 specific strands are shown in FIG. 2, it is well understood in the art that the potential strands that may be isolated number in the thousands. At the time this application is filed, at least 1142 specific and identifiable strands are available for detection and isolation in each sample.

Such isolation processes may comprise the isolating of genetic material based on strands of RNA as identified by a specific gene sequence as described above. Additionally, the isolation of genetic material may be based upon a gene sequence associated with a gene expression indicative of a disease, a gene sequence associated with a gene expression indicative of a trait, a gene sequence associated with a gene expression indicative of a phenotype, and/or a gene sequence associated with a gene expression indicative of a genotype.

With all strands detected and isolated and identified, each set of strands (i.e., all samples with STRAND A isolations) across all samples may be grouped together for additional association and analysis at step 220. As such, all expressions of STRAND A may be grouped into GROUP A 230, all expressions of STRAND B may be grouped into GROUP B 231 and all expressions of STRAND C may be grouped into GROUP C 232. Such grouping allows for the assimilation of data on an aggregate level based on various gene expressions as compared to a number of aggregate level aspects of assimilated data. Specifically, demographic information about the source of a sample may be associated with each sample.

Additionally, aggregating information associated with each blood sample may be accomplished through the groupings of similar strands. Such aggregating includes associating a blood sample exhibiting an expression of a gene sequence indicative of a first disease with the demographic information about the blood sample, associating a blood sample exhibiting an expression of a gene sequence indicative of a first disease with another blood sample exhibiting an expression of a gene sequence indicative of the first disease, associating a blood sample exhibiting an expression of a gene sequence indicative of a first disease with a blood sample exhibiting an expression of a gene sequence indicative of a second disease, associating a blood sample exhibiting an expression of a gene sequence indicative of a first disease with a treatment associated with the first disease, and associating a blood sample exhibiting an expression of a gene sequence indicative of a first disease with a specific polymorphism.

With any number of associations in place from the groupings, statistical data from the aggregated blood samples based on associations of one blood sample with another may be extrapolated. Such statistical data may include expression rates, inter-related expression rates, etc.

Application of this unique set of probes will offer a low cost genomic assessment of an individual's state of health through a new and useful clinical diagnostic. Additionally, adding or deleting probes that relate to a given disease, as new information presents in the literature may further enhance the benefits of the clinical diagnostic. Adding probe content as information expands is a planned future course of action, as will be appreciated by others in the art. Further yet, the clinical diagnostic may be expanded such that components may be tested as separate, and/or all inclusive tests that address different diseases or lifestyle concerns.

Information that may now be gleaned from the groupings of sets of genetic material may be aggregated into in a computer readable medium accessible by a server computer, e.g., a database. Then such data may be accessed by any connected client computer such that information is provided from the aggregated data to a client computer upon a request from the client computer to the server computer.

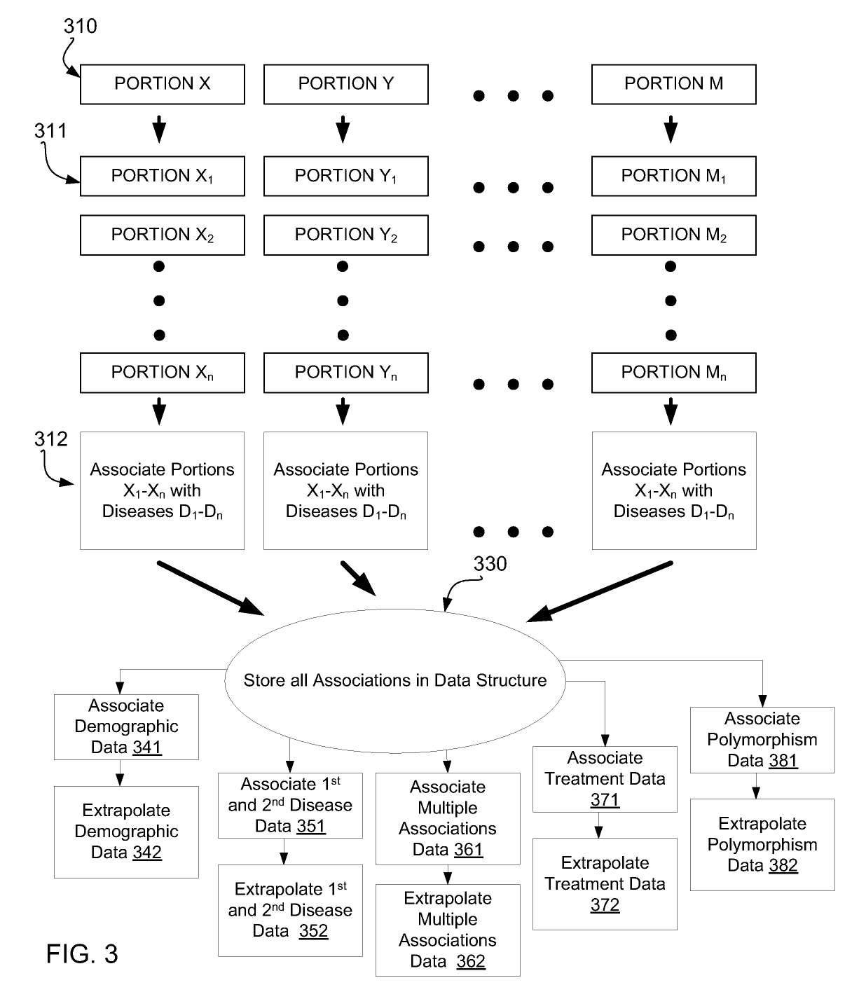

FIG. 3 is a diagrammatic representation of a system and method for establishing a data structure to be used in a broad-based disease association gene transcript test according to an embodiment of an invention disclosed herein.

As samples of genetic material from various sources are gathered, each sample may be identified uniquely by the source of the sample. For example, amongst all samples in FIG. 3, (i.e., Sample X 310 through Sample M), each Sample may be identified uniquely by a tracking identification. For the purposes of the eventual data structure, the first sample may be Sample X, the next may be Sample Y, and so on all the way to the last sample, Sample M. It is understood that these samples may be arranged according to some specific method as described above with respect to FIG. 2 or may also be disposed on a microarray prepared especially for a method and system described herein.

Once all samples are uniquely identified by source, each sample may be further subdivided into specific portions wherein a specific portion may exhibit a specific genetic expression as described above. As used herein, a portion refers to any amount of a genetic material sample that exhibits a specific genetic expression. Portion does not, in any manner, denote a specific amount or quantity of genetic material. As such, each sample may have a very large number of portions, such that each one exhibits a specific genetic expression.

In building a data structure, each portion may be further identified as exhibiting one specific gene expression (or not expressing the gene, as the case may be) at aggregate step 311. Thus, Portion X₁ may be identified as having a first specific nucleotide sequence, portion X₂ may be identified as having a second specific nucleotide sequence and so on until the last portion is identified as having an nth specific nucleotide sequence. With the identification of each portion as containing one of 1^{st}-n^{th} specific nucleotide sequences, the association of the portions with the source (i.e., Sample X) is maintained. A similar portioning of Samples Y through M also maintains the specific association with the source sample. That is, Sample Y is portioned into portion Y₁ through Yₙ each uniquely exhibiting the specific 1^{st} through n^{th} nucleotide sequence respectively. This portioning and association process occurs for all samples through the M^{th} sample.

Next, at aggregate step 312, each portion is associated with a respective disease. That is portion X₁-Xₙ is associated with disease D₁-Dₙ such that each disease that is associated with each portion corresponds uniquely with the specific nucleotide sequence exhibited by the portion. Similarly, portions Y₁-Yₙ are associated with diseases D₁-Dₙ all the way through the M^{th} set of portions wherein portions M₁-Mₙ are associated with diseases D₁-Dₙ, respectively.

With each portion of each sample associated with a specific disease, all broad-based diseased association gene transcript data may be stored in a single data structure 330. With such a data structure in place a number of different associations and data trends may be extrapolated.

For example, if demographics data about the source of the sample was collected at the same time that the sample was collected, the demographics data may also be associated with the expression of specific diseases by associating the demographics data with the portions of each sample exhibiting an expression for such a genetic disease. Then, with these data associations in place within the data structure, such associative data may be extrapolated that encompasses a first disease associated with a portion of a sample with the demographic information about the source of the sample. In the aggregate, specific trends about demographic data and specific diseases may be garnered.

As another example, additional trend data may be garnered by associating a portion of a sample from a first source exhibiting the specific gene expression indicative of a first disease with a portion of a sample from the first source exhibiting the specific gene expression indicative of a second disease. Then, with these associations in place additional trend data may be garnered by extrapolating associative data encompassing a portion of a sample from a first source exhibiting the specific gene expression indicative of a first disease with a portion of a sample from the first source exhibiting the specific gene expression indicative of a second disease. Similarly, such trend data may be garnered by associating specific polymorphisms with specific portions exhibiting such nucleotide sequences associated with the polymorphisms.

Additional information about multiple disease associations may be garnered by associating the portions from the first sample respectively exhibiting specific gene expressions associated with the first and second disease with a portion of a sample from a second source exhibiting the specific gene expressions associated with either the first or the second disease. With these associations, one may extrapolate associative data regarding a portion of a sample from a first source exhibiting the specific gene expression indicative of a first disease, a portion of a sample from the first source exhibiting the specific gene expression indicative of a second disease, and a portion of a sample from a second source exhibiting the specific gene expressions associated with either the first or the second disease in an effort to yield additional trend data.

As yet another example, treatment data may be expressed by associating a portion of a sample from a first source exhibiting the specific gene expression indicative of a first disease with a treatment linked to the first disease. Further, such treatment data may also be extrapolated from such associative that encompasses a portion of a sample from a first source exhibiting the specific gene expression indicative of a first disease with a treatment linked to the first disease.

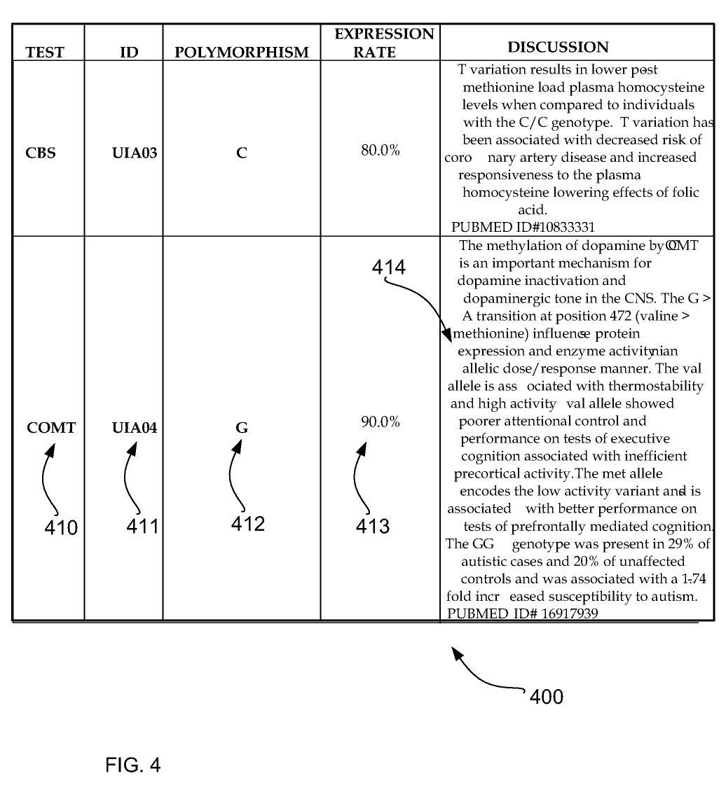

FIG. 4 shows a typical arrangement of data that may be associated in a database of information derived from a broad-based disease association gene transcript test according to an embodiment of an invention disclosed herein. The data associated with the portions of genetic material stemming from traceable samples may be arranged in a data structure 400 according to FIG. 4. In FIG. 4, the data structure may associate a specific test 410, an ID 411, a polymorphism 412, an expression ratio 413, and a discussion 414.

The specific test 410 may typically comprise a known set of nucleotide sequences in which one should examine to determine the presence or non-existence of specific genetic disease or genetic disorder. Based on the polymorphism 412, and ratio 413, the interpretation 414 will indicate the possibilities for diagnosis, or suggest treatment for a specific illness.

The ID 411 may typically comprise the unique identification measure that removes individual identity and replaces it with associative phenotypic characteristics.

The Polymorphism 412 may typically refer to the specific nucleotide that is present for the sample analyzed and may be associated with the presence of a disease. That is, in the specific nucleotide sequence identified in the polymorphism 412, relates to the proportion of analyzed genomic sequences that result from the processing of the test for each individual.

Finally, the data structure may also include a discussion 414 that is obtained from clinically relevant understanding from sources of peer reviewed literature and published clinical studies.

With at least some of these data sets in a data structure, a broad-based disease association gene transcript test data structure may be realized. Such a data structure may be characterized by a first tangible (i.e., fixed in some tangible medium) data set operable to store resulting expression data isolated from genetic material from a specific source, the gene expression associated with a first disease, a second tangible data set operable to store an identification of the source and associated with the first tangible data set, and a third tangible data set operable to store at least one other association with a second disease, the second disease associated with a second gene expression.

Additional data sets may include a fourth tangible data set operable to store an identification of a specific test associated with the first disease, a fifth tangible data set operable to store an expression rate associated with the first disease and associated with the first gene expression, and a sixth tangible data set operable to store a discussion associated with the first disease and associated with the first gene expression. Such a data structure may be realized in a fixed computer-readable medium, such as a database, or may be fixed to another medium such as a substrate hosting a microarray of genetic samples.

A specific combination of nucleic acid sequences taken from isolated regions of the human genome may be reflected as custom content on a platform independent gene expression microarray. A complete list of nucleic acid sequences form the elements analyzed within this human genome examination may form the basic nature of a gene transcript test, which is typically intended for clinical use in effectively detecting transcribed alterations in the genetic code that have a documented relationship with disease, association with therapeutic response, and/or treatment for disease. The content of the test may assess. RNA through quantitative (measurement and assessment of transcript present within the tissue) and qualitative (measurement of genomic regions) means.

This nucleic acid array may be comprised of probe sequences isolated to detect regions within a given gene that most effectively indicate expression levels and that represent polymorphic sections indicating which sequence from the genome an individual is actually expressing. The nucleic acid sequences deemed present in the amplified portions of a sample isolated from standard blood draw and/or disease affected tissue, may be detected by hybridizing the amplified portions to the array and analyzing a hybridization pattern resulting from the hybridization.

Association of test results with claims of clinical relevance may be assimilated and documented as conclusions formed through a comprehensive compilation of peer-reviewed literature (or other periodic update). Ongoing modifications to these claims may be performed through quarterly protocol assessment and maintenance of a peer-to-peer physician support network supported through existing and impending corporate associations.

Paper reporting of the test results may indicate the outcome from a subset of 1 to 50 genetic sequences. Additional reporting for at least 1142 remaining sequences may be made available through alternative measures. These measures may enable physicians to access their patient's information relative to all other patients having ordered the test through a variety of associative clustering methods (hierarchical, divisive, and associative). The concept of creating real-time genotype/phenotype association accessible to physician/physician networks may be further promoted as a desired goal. Physicians will be able to analyze their own patient's data relative to all other data existing individuals who have had the test performed.

Examples of polymorphisms assessed may be single nucleotide polymorphisms (SNPs), deletions, and/or deletion insertion sequences. Further, the polymorphisms predicted to be present in the amplified portions may already be determined. Further yet, the nucleic acid sample may be genomic DNA, cDNA, cRNA, RNA, total RNA or mRNA. With these variations, the SNP, deletion, or insertion may be associated with a disease, the efficacy of a drug, and/or associated with predisposition towards/against development of aforementioned ailment(s). Typically, output data may be packaged in a computer-readable medium (e.g., a CD or DVD) and delivered to a customer, such as a subscribing physician.

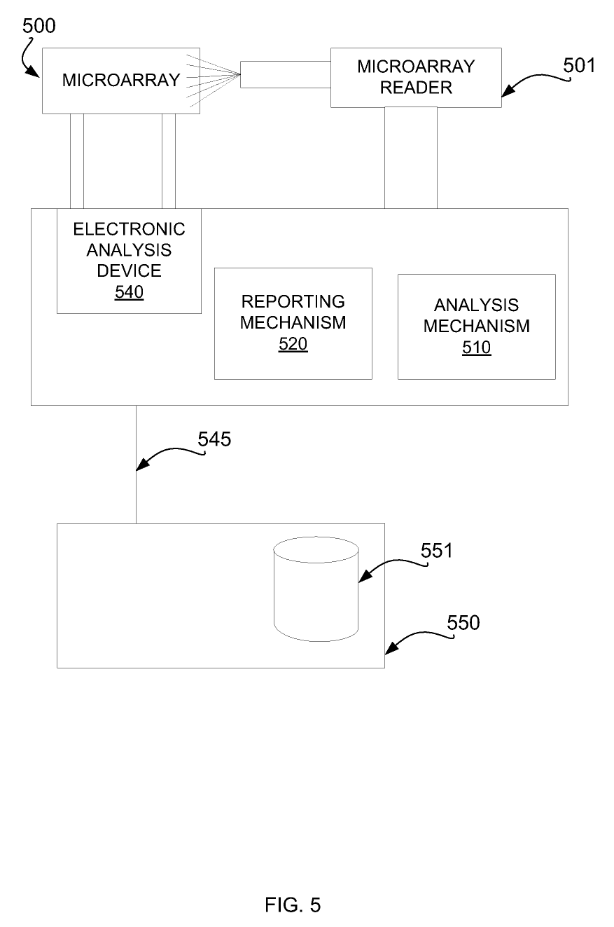

FIG. 5 shows a diagrammatic representation of a method and system for establishing a broad-based disease association gene transcript test according to an embodiment of an invention disclosed herein. In this embodiment, a microarray 500 may be characterized by an arrangement of different identified gene expressions based upon an association with each sample. Several other arrangements of data exists as other embodiments as well. As such, depending on the known arrangement of samples, specific patterns of the presence of phenotypes or lack thereof determine the type of information to be garnered from each prepared microarray 500. As a result of this embodiment, specific patterns emerge indicating a likelihood of occurrence of SNPs, insertions, or deletions in various regions.

Such patterns may be read by a microarray reader 501. The microarray reading device typically includes a microarray station 502 operable to view a microarray 500. As briefly discussed above, a typical microarray 500 will include a plurality of deposit wells suitable for hosting samples of genetic material. The wells disposed on a substrate may be arranged such that each row is suited for hybridizing a genetic material sample such that a unique gene expression may be identified (i.e., one gene per row). Further, each column is suited for having each sample in each row in the column that is associated with a single source of genetic material (i.e., one person per column).

The microarray reader 501 may also typically include an analysis mechanism 510 operable to analyze a pattern displayed on the microarray 500 and a reporting mechanism 520 operable to deliver a report of the analysis. Additionally, an interface 545 to a computer system 550 may allow a reported analysis to be displayed on a display (not shown) and/or stored in a computer-readable medium 551. The microarray reader 501 may also have an electronic microarray assessment apparatus 540 operable to determine a pattern of gene expression from a series of electrical pulses sent to and received from the stationed microarray 500.

Microarrays 500 are quite useful is mapping or “expressing” data about the makeup of the genetic material disposed thereon. Applications of these microarrays 500 include the following. Messenger RNA or Gene Expression Profiling—monitoring expression levels for thousands of genes simultaneously is relevant to many areas of biology and medicine, such as studying treatments, disease, and developmental stages. For example, microarrays 500 can be used to identify disease genes by comparing gene expression in diseased and normal cells. Comparative Genomic Hybridization—this typical use comprises assessing large genomic rearrangements within a single species. SNP detection—looking for Single Nucleotide Polymorphism in the genome of populations of a species. Chromatin Immunoprecipitation Studies—determining protein binding site occupancy throughout the genome, employing chip-on-chip technology. Other uses for microarrays 500 are known and/or contemplated but not discussed herein for brevity.

With such a microarray 500 available for analysis and coupled with multiple additional prepared microarrays, broad-based data about the occurrence or absence of diseases and/or specific gene sequences begins to emerge. The microarray 500 may be scanned and intensity data extracted to associate presence/absence of genetic material in the original sample. This data may be assimilated in a large database of information together with additional information such as diagnosis and treatment information, to provide a multitude of information about a large number of data sets. As the data is assimilated, a comprehensive literature search offering substantiated associations of disease with gene sequence alterations may be provided. The data are rendered anonymous and uploaded into a central repository that allows cross-sample comparison and ultimately, earlier detection of disease.

While the subject matter discussed herein is susceptible to various modifications and alternative constructions, certain illustrated embodiments thereof are shown in the drawings and have been described above in detail. It should be understood, however, that there is no intention to limit the claims to the specific forms disclosed, but on the contrary, the intention is to cover all modifications, alternative constructions, and equivalents falling within the spirit and scope of the claims.

What is claimed is:

1. A method for assembling gene transcript data from a plurality of genetic material sources, the method comprising:
obtaining a sample of genetic material from a plurality of sources of genetic material;
for each sample, isolating portions of each sample such that each isolated portion exhibits a specific gene expression associated with one of a plurality of diseases, each isolated portion corresponding uniquely with an associated disease;
associating each portion with its source;
associating each portion with the corresponding disease; and
storing each association in a data structure.

2. The method of claim 1, further comprising associating demographic data about the source of each sample with each portion of each sample.

3. The method of claim 2, further comprising extrapolating associative data from the data structure, the associative data encompassing a first disease associated with a portion of a sample with the demographic information about the source of the sample.

4. The method of claim 1, further comprising associating a portion of a sample from a first source exhibiting the specific gene expression indicative of a first disease with a portion of a sample from the first source exhibiting the specific gene expression indicative of a second disease.

5. The method of claim 4, further comprising extrapolating associative data from the data structure, the associative data encompassing a portion of a sample from a first source exhibiting the specific gene expression indicative of a first disease with a portion of a sample from the first source exhibiting the specific gene expression indicative of a second disease.

6. The method of claim 4, further comprising associating the portions from the first sample respectively exhibiting specific gene expressions associated with the first and second disease with a portion of a sample from a second source exhibiting the specific gene expressions associated with either the first or the second disease.

7. The method of claim 6, further comprising extrapolating associative data from the data structure, the associative data encompassing:
a portion of a sample from a first source exhibiting the specific gene expression indicative of a first disease:
a portion of a sample from the first source exhibiting the specific gene expression indicative of a second disease; and
a portion of a sample from a second source exhibiting the specific gene expressions associated with either the first or the second disease

10. The method of claim 1, further comprising associating a portion of a sample from a first source exhibiting the specific gene expression indicative of a first disease with a treatment linked to the first disease.

11. The method of claim 10, further comprising extrapolating associative data from the data structure, the associative data encompassing a portion of a sample from a first source exhibiting the specific gene expression indicative of a first disease with a treatment linked to the first disease.

12. The method of claim 1, further comprising associating a portion of a sample from a first source exhibiting the specific gene expression indicative of a first disease with a specific polymorphism.

13. The method of claim 12, further comprising extrapolating associative data from the data structure, the associative data encompassing a portion of a sample from a first source exhibiting the specific gene expression indicative of a first disease with a specific polymorphism.

14. A data structure, comprising:
a first data set fixed in a tangible medium operable to store a gene expression isolated from genetic material from a specific source, the gene expression associated with a first disease;
a second data set fixed in a tangible medium operable to store an identification of the source and associated with the first tangible data set; and
a third data set fixed in a tangible medium operable to store at least one other association with a second disease, the second disease associated with a second gene expression.

15. The data structure of claim 14, further comprising a fourth data set fixed in a tangible medium operable to store an identification of a specific test associated with the first disease.

16. The data structure of claim 15, further comprising a fifth data set fixed in a tangible medium operable to store an expression rate associated with the first disease and associated with the first gene expression.

17. The data structure of claim 16, further comprising a sixth data set fixed in a tangible medium operable to store a discussion associated with the first disease and associated with the first gene expression.

18. A data structure reading device, comprising:
a microarray station operable to analyze a microarray comprising:
a plurality of deposit wells suitable for hosting samples of genetic material;
each row suited for hybridizing a genetic material sample such that a unique gene expression may be identified;
each column suited for having each sample in each row in the column be associated with a single source of genetic material;
an analysis mechanism operable to analyze at least one pattern evident from the microarray; and
a reporting mechanism operable to deliver a report of the analysis.

19. The data structure reading device of claim 18, further comprising an interface to a computer system such that the reported analysis may be displayed on a display and stored in a computer-readable medium.

20. The data structure reading device of claim 18, wherein the analysis mechanism further comprises an electronic microarray assessment apparatus operable to determine a pattern of gene expression from a series of electrical pulses sent to and received from the microarray.
